(19) United States
(12) Patent Application Publication
(10) Pub. No.: US 2011/0020325 A1
(43) Pub. Date: Jan. 27, 2011

(54) METHODS FOR REDUCING GRANULOMATOUS INFLAMMATION

(76) Inventors: Eugene D. Kwon, Rochester, MN (US); John C. Cheville, Pine Island, MN (US); Thomas J. Sebo, Rochester, MN (US); Brant A. Inman, Edmonton (CA)

Correspondence Address: FISH & RICHARDSON P.C. (TC), PO BOX 1022, MINNEAPOLIS, MN, 55440-1022, US

(21) Appl. No.: 12/920,021

(22) PCT Filed: Feb. 27, 2009
(86) PCT No.: PCT/US09/35495

§ 371 Date: Oct. 14, 2010

Related U.S. Application Data
(60) Provisional application No. 61/032,706, filed on Feb. 29, 2008.

(51) Int. Cl. A61K 39/395; A61K 31/713; A61P 31/06; A61P 31/08

(52) U.S. Cl. 424/130.1; 514/44 A

(57) ABSTRACT

This document provides methods and materials for reducing bacterial induced granulomatous inflammation in a mammal using agents that reduce B7-H1 expression or activity.

### CROSS-REFERENCE TO RELATED APPLICATIONS

This application claims the benefit of U.S. Provisional Application Ser. No. 61/032,706, filed on Feb. 29, 2008. The disclosure of the prior application is incorporated by reference in its entirety.

### TECHNICAL FIELD

This document relates to materials and methods for reducing granulomatous inflammation in a mammal, and more particularly to materials and methods for reducing granulomatous inflammation using agents that inhibit coinhibitory molecules such as B7-H1 or B7-H4.

### BACKGROUND

Granulomas are a characteristic feature of many human pathologies including a wide variety of infectious diseases, idiopathic autoimmune disorders, vasculitic disorders, malignancies and wound healing problems. These disorders share the presence of a chronic inflammatory state, the etiology of which may be evident or not, that leads to a granulomatous inflammatory response. The physiologic processes that regulate the formation of granulomata have not been completely elucidated but appear to involve a complex interplay between T lymphocytes and macrophages (or macrophage-like cells such as giant cells and epithelioid cells). Other cell types, such as dendritic cells and B lymphocytes, also may be involved.

Some hosts have the ability to dissolve and clear their granulomas while other hosts seem to preferentially generate destructive fibrotic and necrotic granulomatous reactions. Dheda et al., J. Infect. Dis. (2005) 192(7):1201-1209. It appears that the granulomatous immune response can differ not only from host to host but also from pathogen to pathogen. This heterogeneity is illustrated by the spectrum of disease that is observed clinically in leprosy, a serious granulomatous disease induced by Mycobacterium leprae. Some patients suffer from multibacillary Hansen's disease (lepromatous leprosy), an extremely disfiguring form the disease, while others have the more benign paucibacillary (tuberculoid) form of leprosy that is characterized by hypopigmented skin macules. Britton et al., Lancet (2004), 363(9416):1209-1219. This heterogeneity is thought to be the result of differences in the host immune response to the pathogen.

### SUMMARY

This document provides materials and methods for reducing granulomatous inflammation in a mammal (e.g., a human). For example, the document provides materials and methods for reducing bacterial induced granulomatous inflammation in a mammal such as granulomatous inflammation resulting from a Mycobacterium infection (e.g., Mycobacterium tuberculosis, Mycobacterium leprae, Mycobacterium lepromatosis, or Mycobacterium bovis strain bacille Calmette-Guérin (BCG) infection). The methods can include administering to the mammal an agent that reduces B7-H1 or B7-H4 expression or activity. The agent can be an antibody, an antisense oligonucleotide, or double-stranded small interfering RNA. The agent can be administered locally to the granulomatous inflammation.

his document also provides the use of agent that reduces B7-H1 or B7-H4 expression or activity in the manufacture of a medicament for reducing granulomatous inflammation in a mammal (e.g., a human). Methods of manufacturing medicaments using such agents are well known to persons skilled in the art of medicine and pharmacy. In some embodiments, this document provides a use wherein the granulomatous inflammation is bacterial induced (e.g., from a Mycobacterium tuberculosis, Mycobacterium leprae, Mycobacterium lepromatosis, or Mycobacterium bovis strain BCG infection).

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and from the claims.

### DESCRIPTION OF DRAWING

FIG. 1A is a low-power (2.5×) image of a PD-L1 positive BCG granuloma and FIG. 1B is a high-power (40×) image of PD-L1 positive BCG granuloma from patients with recurrent bladder cancer.

### DETAILED DESCRIPTION

In general, the present application provides methods and materials for reducing granulomatous inflammation in a mammal. As used herein, “granulomatous inflammation” refers to a proliferative inflammation characterized by the formation of granulomas. The term “granuloma” refers to a chronic inflammatory lesion characterized by large numbers of cells of various types (macrophages, lymphocytes, fibroblasts, giant cells), some degrading and some repairing the tissues. Granulomatous inflammation is associated with a wide variety of human pathologies, including, for example, idiopathic autoimmune disorders, vasculitic disorders, infectious diseases, malignancies, and wound healing problems (Table 1). Reducing granulomatous inflammation can include reducing the severity of the inflammation, slowing progression of the inflammation, or preventing formation of fibrotic or necrotic tissue.

TABLE 1
Diseases associated with granulomatous inflammation

| | Diagnostic entity |
|---|---|
| Idiopathic/Immune | Churg-Strauss syndrome |
| | Crohn's disease |
| | Giant cell myocarditis |
| | Granulomatous hepatitis |
| | Malakoplakia |
| | Primary biliary cirrhosis |
| | Ulcerative colitis |
| | Wegeners's granulomatosis |
| | Whipple's disease |
| Cutaneous | Actinic granuloma |
| | Dermatophytic granuloma |
| | Giant cell granuloma |
| | Granuloma annulare |
| | Pyogenic granuloma |
| Infectious | |
| Bacterial | Cat scratch disease |
| | Granuloma inguinale |
| | Leprosy |
| | Lyme disease |
| | Lymphogranuloma venereum |
| | Q fever |
| | Syphilis |
| | Tuberculosis |
| | Xanthogranulomatous |
| | pyelonephritis |
| Fungal | Aspergillosis |
| | Blastomycosis |
| | Coccidioidomycosis |
| | Cryptococcosis |
| | Histoplasmosis |
| | Pneumocystic carinii |
| | Zygomycosis |
| Parasitic | Leischmaniasis |
| | Malaria |
| | Schistosomiasis (bilharzia) |
| Foreign body reactions | Spindle cell nodule |
| | Inflammatory pseudotumor |
| Neoplastic | Bladder: squamous cell |
| | carcinoma |
| | Breast: ductal carcinoma |
| | Colon: adenocarcinoma |
| | Lung: squamous cell carcinoma |
| | Lymphoid: lymphoma |
| | Myeloid: Langerhans cell |
| | histiocytosis |
| | Ovary: dysgerminoma |
| | Stomach: inflammatory fibroid |
| | polyp |
| | Testis: seminoma |

B7-H1 immunostaining has been observed in a number of histologic granulomatas in a variety of tissue specimens. In particular, positive B7-H1 immunostaining was nearly ubiquitous in the macrophages and epithelioid cells of the granulomas. Lymphocytes bordering the granuloma also were positive in a number of cases. As described herein, B7-H1 and other coinhibitory molecules such as B7-H4 can be therapeutically targeted to reduce granulomatous inflammation and improve a variety of granulomatous disorders (e.g., the diseases set forth in Table 1). For example, B7-H1 and/or B7-H4 can be targeted to treat bacterial induced diseases such as leprosy (Mycobacterium leprae or Mycobacterium lepromatosis), tuberculosis (Mycobacterium tuberculosis), syphilis (T. pallidum pallidum), cat scratch disease (Bartonella henselae), lyme's disease (Borrelia burgdorferi, Borrelia afzelii, or Borrelia garinii), granuloma inguinale (Calymmatobacterium granulomatis), lymphogranuloma venereum (serovars L1, L2, or L3 of Chlamydia trachomatis), Q fever (Coxiella burnetii), or xanthogranulomatous pyelonephritis (Proteus, E. coli, or Pseudomonas). Without being bound to a particular mechanism, B7-H1 and other coinhibitory molecules such as B7-H4 may play a role in initiating and maintaining immunosuppressive phenomena in granulomatous disorders.

The term “B7-H1” refers to B7-H1 from any mammalian species and the term “hB7-H1” refers to human B7-H1. Further details on B7-H1 polypeptides and nucleic acids are provided in U.S. Pat. No. 6,803,192, the disclosure of which is incorporated herein by reference in its entirety. The nucleotide and amino acid sequences of hB7-H1 can be found in GenBank under Accession Nos. AF177937 and AAF25807, respectively. B7-H1 (also known as programmed death (PD)-L1 and CD274) is a negative regulator of T cell-mediated immunity. See, Dong et al. (1999) Nat. Med. 5, 1365-1369; Dong et al. (2002) Nat. Med. 8, 793-800; and Thompson et al. (2004) Proc. Natl. Acad. Sci. USA 101, 17174-17179.

The term “B7-H4” refers to B7-H4 from any mammalian species and the term “hB7-H4” refers to human B7-H4. Further details on B7-H4 polypeptides and nucleic acids are provided in U.S. Pat. No. 6,891,030, the disclosure of which is incorporated herein by reference in its entirety. The nucleotide and amino acid sequences of hB7-H4 can be found in GenBank under Accession Nos. AY280972 and AAP37283, respectively. B7-H4 is a negative regulator of T cell-mediated immunity.

Any agent that reduces B7-H1 or B7-H4 expression or activity can be used to reduce granulomatous inflammation in a mammal (e.g., in a human patient). For example, anti-B7-H1 or anti-B7-H4 antibodies can be used to reduce granulomatous inflammation in a mammal. In some cases, antisense oligonucleotides, siRNA molecules, RNAi constructs, or PNA oligomers can be designed and used to reduce the level of B7-H1 or B7-H4 polypeptides expressed. In addition, agents (e.g., small molecule inhibitors) that bind to a B7-H1 or B7-H4 polypeptide and inhibit a B7-H1 or B7-H4 polypeptide activity can be used to reduce granulomatous inflammation in a mammal. Such agents can be identified using any appropriate method. For example, an organic small molecule capable of inhibiting a B7-H1 or B7-H4 polypeptide activity can be identified by screening a small molecule library for molecules having the ability to bind to a B7-H1 or B7-H4 polypeptide and the ability to reduce granulomatous inflammation in a manner dependent on B7-H1 or B7-H4 polypeptide expression.

As described herein, an agent that reduces B7-H1 or B7-H4 expression or activity can be an anti-B7-H1 or B7-H4 antibody. For example, in one embodiment, this document provides methods for reducing granulomatous inflammation in a mammal by administering an anti-B7-H1 or anti-B7-H4 antibody to the mammal.

The term “antibody” as used herein refers to intact antibodies as well as antibody fragments that retain some ability to bind an epitope. Such fragments include, without limitation, Fab, F(ab′)2, and Fv antibody fragments. The term “epitope” refers to an antigenic determinant on an antigen to which the paratope of an antibody binds. Epitopic determinants usually consist of chemically active surface groupings of molecules (e.g., amino acid or sugar residues) and usually have specific three dimensional structural characteristics as well as specific charge characteristics.

The antibodies provided herein can be any monoclonal or polyclonal antibody having binding affinity for a B7-H1 or B7-H4 polypeptide (e.g., an hB7-H1 or hB7-H4 polypeptide). In some cases, an anti-B7-H1 or anti-B7-H4 antibody can exhibit little, or no, detectable cross reactivity with polypeptides sharing no homology with a B7-H1 or B7-H4 polypeptide.

Anti-B7-H1 or anti-B7-H4 antibodies can be obtained from a commercial vender. In some cases, an anti-B7-H1 or anti-B7-H4 antibody provided herein can be prepared using any appropriate method. See, for example, Dong et al. (2002) Nature Med. 8:793-800. For example, any substantially pure B7-H1 or B7-H4 polypeptide, or fragment thereof, can be used as an immunogen to elicit an immune response in an animal such that specific antibodies are produced. Thus, an hB7-H1 or hB7-H4 polypeptide or a fragment thereof can be used as an immunizing antigen. In addition, the immunogen used to immunize an animal can be chemically synthesized or derived from translated cDNA. Further, the immunogen can be conjugated to a carrier polypeptide, if desired. Commonly used carriers that are chemically coupled to an immunizing polypeptide include, without limitation, keyhole limpet hemocyanin (KLH), thyroglobulin, bovine serum albumin (BSA), and tetanus toxoid.

The preparation of polyclonal antibodies is well-known to those skilled in the art. See, e.g., Green et al., Production of Polyclonal Antisera, in IMMUNOCHEMICAL PROTOCOLS (Manson, ed.), pages 1 5 (Humana Press 1992) and Coligan et al., Production of Polyclonal Antisera in Rabbits, Rats, Mice and Hamsters, in CURRENT PROTOCOLS IN IMMUNOLOGY, section 2.4.1 (1992). In addition, those of skill in the art will know of various techniques common in the immunology arts for purification and concentration of polyclonal antibodies, as well as monoclonal antibodies (Coligan, et al., Unit 9, Current Protocols in Immunology, Wiley Interscience, 1994).

The preparation of monoclonal antibodies also is well-known to those skilled in the art. See, e.g., Kohler & Milstein, Nature 256:495 (1975); Coligan et al., sections 2.5.1 2.6.7; and Harlow et al., ANTIBODIES: A LABORATORY MANUAL, page 726 (Cold Spring Harbor Pub. 1988). Briefly, monoclonal antibodies can be obtained by injecting mice with a composition comprising an antigen, verifying the presence of antibody production by analyzing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well established techniques. Such isolation techniques include affinity chromatography with Protein A Sepharose, size exclusion chromatography, and ion exchange chromatography. See, e.g., Coligan et al., sections 2.7.1 2.7.12 and sections 2.9.1 2.9.3; Barnes et al., Purification of Immunoglobulin G (IgG), in METHODS IN MOLECULAR BIOLOGY, VOL. 10, pages 79 104 (Humana Press 1992).

In addition, methods of in vitro and in vivo multiplication of monoclonal antibodies are well known to those skilled in the art. Multiplication in vitro can be carried out in suitable culture media such as Dulbecco's Modified Eagle Medium or RPMI 1640 medium, optionally replenished by mammalian serum such as fetal calf serum, or trace elements and growth sustaining supplements such as normal mouse peritoneal exudate cells, spleen cells, and bone marrow macrophages. Production in vitro provides relatively pure antibody preparations and allows scale up to yield large amounts of the desired antibodies. Large scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells (e.g., osyngeneic mice) to cause growth of antibody producing tumors. Optionally, the animals are primed with a hydrocarbon, especially oils such as pristane (tetramethylpentadecane) prior to injection. After one to three weeks, the desired monoclonal antibody is recovered from the body fluid of the animal.

In some cases, the antibodies provided herein can be made using non-human primates. General techniques for raising therapeutically useful antibodies in baboons can be found, for example, in Goldenberg et al., International Patent Publication WO 91/11465 (1991) and Losman et al., Int. J. Cancer, 46:310 (1990).

In some cases, the antibodies can be humanized monoclonal antibodies. Humanized monoclonal antibodies can be produced by transferring mouse complementarity determining regions (CDRs) from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions when treating humans. General techniques for cloning murine immunoglobulin variable domains are described, for example, by Orlandi et al., Proc. Nat'l. Acad. Sci. USA, 86:3833 (1989). Techniques for producing humanized monoclonal antibodies are described, for example, by Jones et al., Nature, 321:522 (1986); Riechmann et al., Nature, 332:323 (1988); Verhoeyen et al., Science, 239:1534 (1988); Carter et al., Proc. Nat'l. Acad. Sci. USA, 89:4285 (1992); Sandhu, Crit. Rev. Biotech., 12:437 (1992); and Singer et al., J. Immunol., 150:2844 (1993).

Antibodies provided herein can be derived from human antibody fragments isolated from a combinatorial immunoglobulin library. See, for example, Barbas et al., METHODS: A COMPANION TO METHODS IN ENZYMOLOGY, VOL. 2, page 119 (1991) and Winter et al., Ann. Rev. Immunol., 12: 433 (1994). Cloning and expression vectors that are useful for producing a human immunoglobulin phage library can be obtained, for example, from STRATAGENE Cloning Systems (La Jolla, Calif.).

In addition, antibodies provided herein can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens and can be used to produce human antibody secreting hybridomas. Methods for obtaining human antibodies from transgenic mice are described by Green et al., Nature Genet., 7:13 (1994); Lonberg et al., Nature, 368:856 (1994); and Taylor et al., Int. Immunol., 6:579 (1994).

Antibody fragments can be prepared by proteolytic hydrolysis of an intact antibody or by the expression of a nucleic acid encoding the fragment. Antibody fragments can be obtained by pepsin or papain digestion of intact antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. In some cases, an enzymatic cleavage using pepsin can be used to produce two monovalent Fab′ fragments and an Fc fragment directly. These methods are described, for example, by Goldenberg (U.S. Pat. Nos. 4,036,945 and 4,331,647). See, also, Nisonhoff et al., Arch. Biochem. Biophys., 89:230 (1960); Porter, Biochem. J., 73:119 (1959); Edelman et al., METHODS IN ENZYMOLOGY, VOL. 1, page 422 (Academic Press 1967); and Coligan et al. at sections 2.8.1 2.8.10 and 2.10.1 2.10.4.

Other methods of cleaving antibodies, such as separation of heavy chains to form monovalent light heavy chain fragments, further cleavage of fragments, or other enzymatic, chemical, or genetic techniques may also be used provided the fragments retain some ability to bind (e.g., selectively bind) its epitope.

The antibodies provided herein can be substantially pure. The term “substantially pure” as used herein with reference to an antibody means the antibody is substantially free of other polypeptides, lipids, carbohydrates, and nucleic acid with which it is naturally associated in nature. Thus, a substantially pure antibody is any antibody that is removed from its natural environment and is at least 60 percent pure. A substantially pure antibody can be at least about 65, 70, 75, 80, 85, 90, 95, or 99 percent pure.

In other embodiments, nucleic acid based methods, including antisense RNA, ribozyme directed RNA cleavage, or post-transcriptional gene silencing (PTGS), e.g., double-stranded small interfering RNA (siRNA) can be used to reduce B7-H1 or B7-H4 gene expression. For example, in one embodiment, this document provides methods for reducing granulomatous inflammation in a mammal by administering one or more antisense oligonucleotides to the mammal (e.g., a human). Antisense oligonucleotides typically are at least 8 nucleotides in length. For example, an antisense oligonucleotide can be about 8, 9, 10-20 (e.g., 11, 12, 13, 14, 15, 16, 17, 18, 19, or 20 nucleotides in length), 15 to 20, 18-25, or 20-50 nucleotides in length. In other embodiments, antisense molecules can be used that are greater than 50 nucleotides in length, including the full-length sequence of a B7-H1 or B7-H4 mRNA. As used herein, the term “oligonucleotide” refers to an oligomer or polymer of ribonucleic acid (RNA) or deoxyribonucleic acid (DNA) or analogs thereof. Nucleic acid analogs can be modified at the base moiety, sugar moiety, or phosphate backbone to improve, for example, stability, hybridization, or solubility of a nucleic acid. Modifications at the base moiety include substitution of deoxyuridine for deoxythymidine, and 5-methyl-2′-deoxycytidine and 5-bromo-2′-deoxycytidine for deoxycytidine. Other examples of nucleobases that can be substituted for a natural base include 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 8-azaguanine and 8-azaadenine, 7-deazaguanine and 7-deazaadenine and 3-deazaguanine and 3-deazaadenine. Other useful nucleobases include those disclosed, for example, in U.S. Pat. No. 3,687,808.

Modifications of the sugar moiety can include modification of the 2′ hydroxyl of the ribose sugar to form 2′-O-methyl or 2′-O-allyl sugars. The deoxyribose phosphate backbone can be modified to produce morpholino nucleic acids, in which each base moiety is linked to a six-membered, morpholino ring, or peptide nucleic acids, in which the deoxyphosphate backbone is replaced by a pseudopeptide backbone (e.g., an aminoethylglycine backbone) and the four bases are retained. See, for example, Summerton and Weller (1997) Antisense Nucleic Acid Drug Dev. 7:187-195; and Hyrup et al. (1996) Bioorgan. Med. Chem. 4:5-23. In addition, the deoxyphosphate backbone can be replaced with, for example, a phosphorothioate or phosphorodithioate backbone, a phosphoroamidite, or an alkyl phosphotriester backbone. See, for example, U.S. Pat. Nos. 4,469,863, 5,235,033, 5,750,666, and 5,596,086 for methods of preparing oligonucleotides with modified backbones.

Antisense oligonucleotides also can be modified by chemical linkage to one or more moieties or conjugates that enhance the activity, cellular distribution or cellular uptake of the oligonucleotide. Such moieties include but are not limited to lipid moieties (e.g., a cholesterol moiety); cholic acid; a thioether moiety (e.g., hexyl-S-tritylthiol); a thiocholesterol moiety; an aliphatic chain (e.g., dodecandiol or undecyl residues); a phospholipid moiety (e.g., di-hexadecyl-rac-glycerol or triethyl-ammonium 1,2-di-O-hexadecyl-rac-glycero-3-H-phosphonate); a polyamine or a polyethylene glycol chain; adamantane acetic acid; a palmityl moiety; or an octadecylamine or hexylamino-carbonyl-oxycholesterol moiety. The preparation of such oligonucleotide conjugates is disclosed in, for example, U.S. Pat. Nos. 5,218,105 and 5,214,136.

Methods for synthesizing antisense oligonucleotides are known, including solid phase synthesis techniques. Equipment for such synthesis is commercially available from several vendors including, for example, Applied Biosystems (Foster City, Calif.). Alternatively, expression vectors that contain a regulatory element that directs production of an antisense transcript can be used to produce antisense molecules.

Antisense oligonucleotides can bind to a nucleic acid encoding B7-H1 or B7-H4, including DNA encoding B7-H1 or H4 RNA (including pre-mRNA and mRNA) transcribed from such DNA, and also cDNA derived from such RNA, under physiological conditions (i.e., physiological pH and ionic strength). It is understood in the art that the sequence of an antisense oligonucleotide need not be 100% complementary to that of its target nucleic acid to be hybridizable under physiological conditions. Antisense oligonucleotides hybridize under physiological conditions when binding of the oligonucleotide to the B7-H1 or B7-H4 nucleic acid interferes with the normal function of the B7-H1 or B7-H4 nucleic acid, and non-specific binding to non-target sequences is minimal.

Target sites for B7-H1 or B7-H4 antisense oligonucleotides include the regions encompassing the translation initiation or termination codon of the open reading frame (ORF) of the gene. In addition, the ORF has been targeted effectively in antisense technology, as have the 5′ and 3′ untranslated regions. Furthermore, antisense oligonucleotides have been successfully directed at intron regions and intron-exon junction regions. Further criteria can be applied to the design of antisense oligonucleotides. Such criteria are well known in the art, and are widely used, for example, in the design of oligonucleotide primers. These criteria include the lack of predicted secondary structure of a potential antisense oligonucleotide, an appropriate G and C nucleotide content (e.g., approximately 50%), and the absence of sequence motifs such as single nucleotide repeats (e.g., GGGG runs). The effectiveness of antisense oligonucleotides at modulating expression of a B7-H1 or B7-H4 nucleic acid can be evaluated by measuring levels of the B7-H1 or B7-H4 mRNA or protein (e.g., by Northern blotting, RT-PCR, Western blotting, ELISA, or immunohistochemical staining).

In another method, a ribozyme or catalytic RNA can be used to affect expression of an mRNA, such as a B7-H1 or B7-H4 mRNA. See, U.S. Pat. No. 6,423,885. Ribozymes can be designed to specifically pair with virtually any target RNA and cleave the phosphodiester backbone at a specific location, thereby functionally inactivating the target RNA. Heterologous nucleic acids can encode ribozymes designed to cleave particular mRNA transcripts, thus preventing expression of a polypeptide. Hammerhead ribozymes are useful for destroying particular mRNAs, although various ribozymes that cleave mRNA at site-specific recognition sequences can be used. Hammerhead ribozymes cleave mRNAs at locations dictated by flanking regions that form complementary base pairs with the target mRNA. The sole requirement is that the target RNA contains a 5′-UG-3′ nucleotide sequence. The construction and production of hammerhead ribozymes is known in the art. See, for example, U.S. Pat. No. 5,254,678 and WO 02/46449 and references cited therein. Hammerhead ribozyme sequences can be embedded in a stable RNA such as a transfer RNA (tRNA) to increase cleavage efficiency in vivo. Perriman et al., Proc. Natl. Acad. Sci. USA, 92(13):6175-6179 (1995); de Feyter and Gaudron, Methods in Molecular Biology, Vol. 74, Chapter 43, “Expressing Ribozymes in Plants”, Edited by Turner, P. C., Humana Press Inc., Totowa, N.J. RNA endoribonucleases which have been described, such as the one that occurs naturally in Tetrahymena thermophila, can be useful. See, for example, U.S. Pat. Nos. 4,987,071 and 6,423,885.

In another embodiment, PNA (polyamide nucleic acid or peptide nucleic acid) oligomers can be used to reduce granulomatous inflammation in a mammal. PNA oligomers are modified oligonucleotides in which the phosphodiester backbone of the oligonucleotide is replaced with a neutral polyamide backbone consisting of N-(2-aminoethyl)glycine units linked through amide bonds. See, e.g., Nielsen et al. (1991) Science 254:1497-1500, and Nielsen et al. (1994) Bioconjugate Chem. 5:3-7.

In another embodiment, this document provides methods for reducing granulomatous inflammation in a mammal by administering, to the mammal, nucleic acid that induces RNA interference against nucleic acid encoding a B7-H1 or B7-H4 polypeptide in the mammal. For example, double-stranded small interfering RNA (siRNA) homologous to a B7-H1 or B7-H4 DNA can be used to reduce expression of that DNA. Constructs for siRNA can be constructed as described, for example, in Fire et al. (1998) Nature 391:806-811; Romano and Masino (1992) Mol. Microbiol. 6:3343-3353; Cogoni et al. (1996) EMBO J. 15:3153-3163; Cogoni and Masino (1999) Nature 399:166-169; Misquitta and Paterson (1999) Proc. Natl. Acad. Sci. USA 96:1451-1456; and Kennerdell and Carthew (1998) Cell 95:1017-1026.

The sense and anti-sense RNA strands of siRNA can be individually constructed using chemical synthesis and enzymatic ligation reactions using procedures known in the art. For example, each strand can be chemically synthesized using naturally occurring nucleotides or variously modified nucleotides designed to increase the biological stability of the molecule or to increase the physical stability of the duplex formed between the sense and anti-sense strands, e.g., phosphorothioate derivatives and acridine substituted nucleotides. The sense or anti-sense strand can also be produced biologically using an expression vector into which a target sequence (full-length or a fragment) has been subcloned in a sense or anti-sense orientation. The sense and anti-sense RNA strands can be annealed in vitro before delivery of the dsRNA to cells. Alternatively, annealing can occur in vivo after the sense and anti-sense strands are sequentially delivered to neural cells.

Any appropriate method can be used to deliver nucleic acid such as a B7-H1 or B7-H4 antisense oligonucleotide or a B7-H1 or B7-H4 siRNA construct to a cell. For example, liposomes or lipids can be loaded or complexed with nucleic acid to form nucleic acid-liposome or nucleic acid-lipid complexes. The liposome can be composed of cationic and neutral lipids commonly used to transfect cells in vitro. Cationic lipids can complex (e.g., charge-associate) with negatively charged nucleic acids to form liposomes. Examples of cationic liposomes include lipofectin, lipofectamine, lipofectace, and DOTAP. Procedures for forming liposomes are well known in the art. Liposome compositions can be formed, for example, from phosphatidylcholine, dimyristoyl phosphatidylcholine, dipalmitoyl phosphatidylcholine, dimyristoyl phosphatidylglycerol, or dioleoyl phosphatidylethanolamine. Numerous lipophilic agents are commercially available, including Lipofectin® (Invitrogen/Life Technologies, Carlsbad, Calif.) and Effectene™ (Qiagen, Valencia, Calif.).

In some embodiments, systemic delivery can be optimized using commercially available cationic lipids such as DDAB or DOTAP, each of which can be mixed with a neutral lipid such as DOPE or cholesterol. In some cases, liposomes such as those described by Templeton et al. (Nature Biotechnology, 15:647-652 (1997)) can be used. In other embodiments, polycations such as polyethyleneimine can be used to achieve delivery in vivo and ex vivo (Boletta et al., J. Am Soc. Nephrol. 7: 1728 (1996)).

The mode of delivery can vary with the targeted cell or tissue. For example, nucleic acids can be delivered to lung and liver via the intravenous injection of liposomes since both lung and liver tissue take up liposomes in vivo. In addition, catheterization in an artery upstream of the affected organ can be used to deliver liposomes containing nucleic acid. This catheterization can avoid clearance of the liposomes from the blood by the lungs and/or liver.

Liposomes containing nucleic acid can be administered parenterally, intravenously, intramuscularly, intraperitoneally, transdermally, excorporeally, or topically. The dosage can vary depending on the species, age, weight, condition of the subject, and the particular compound delivered.

In some embodiments, viral vectors can be used to deliver nucleic acid to a desired target cell. Standard molecular biology techniques can be used to introduce a nucleic acid provided herein into one of the many different viral vectors previously developed to deliver nucleic acid to particular cells. These resulting viral vectors can be used to deliver nucleic acid to the targeted cells by, for example, infection.

An agent having the ability to reduce B7-H1 or B7-H4 expression or activity can be administered in amounts and for periods of time that will vary depending upon the nature of the granulomatous inflammation and the mammal's overall condition. Agents designed to reduce B7-H1 or B7-H4 polypeptide expression (e.g., siRNA molecules) can be administered in an amount that effectively reduces production of the targeted B7-H1 or B7-H4 polypeptide. The ability of an agent to effectively reduce production of a B7-H1 or B7-H4 polypeptide can be assessed, for example, by measuring mRNA or polypeptide levels in a mammal before and after treatment. Any appropriate method can be used to measure mRNA and polypeptide levels in tissues or biological samples such as Northern blots, RT-PCR, immunostaining, ELISAs, and radioimmunoassays. Agents designed to inhibit a B7-H1 or B7-H4 polypeptide activity by interacting with a B7-H1 or B7-H4 polypeptide can be administered in an amount that effectively inhibits a B7-H1 or B7-H4 polypeptide activity or reduces granulomatous inflammation. Effective amounts of agents that reduce B7-H1 or B7-H4 expression or activity can be determined by a physician, taking into account various factors that can modify the action of drugs such as overall health status, body weight, sex, diet, time and route of administration, other medications, and any other relevant clinical factors.

Any appropriate method can be used to formulate and subsequently administer a composition containing one or more agents having the ability to reduce B7-H1 or B7-H4 expression or activity. For example, compositions containing one or more agents having the ability to reduce B7-H1 or B7-H4 expression or activity provided herein can be admixed, encapsulated, conjugated, or otherwise associated with other molecules such as, for example, liposomes, receptor targeted molecules, oral formulations, rectal formulations, or topical formulations for assisting in uptake, distribution, and/or absorption.

Compositions containing one or more agents having the ability to reduce B7-H1 or B7-H4 expression or activity provided herein can contain one or more pharmaceutically acceptable carriers. A “pharmaceutically acceptable carrier” is a pharmaceutically acceptable solvent, suspending agent, or any other pharmacologically inert vehicle. Pharmaceutically acceptable carriers can be liquid or solid, and can be selected with the planned manner of administration in mind so as to provide for the desired bulk, consistency, and other pertinent transport and chemical properties. Typical pharmaceutically acceptable carriers include, without limitation, water; saline solution; binding agents (e.g., polyvinylpyrrolidone or hydroxypropyl methylcellulose); fillers (e.g., lactose and other sugars, gelatin, or calcium sulfate); lubricants (e.g., starch, polyethylene glycol, or sodium acetate); disintegrates (e.g., starch or sodium starch glycolate); and wetting agents (e.g., sodium lauryl sulfate).

A composition can be administered by a number of methods depending upon whether local or systemic treatment is desired and upon the area to be treated. Administration can be, for example, topical (e.g., transdermal, ophthalmic, or intranasal); pulmonary (e.g., by inhalation or insufflation of powders or aerosols); oral; or parenteral (e.g., by subcutaneous, intrathecal, intraventricular, intramuscular, or intraperitoneal injection, or by intravenous drip). Administration can be rapid (e.g., by injection) or can occur over a period of time (e.g., by slow infusion or administration of slow release formulations). For treating tissues in the central nervous system, a composition can be administered by injection or infusion into the cerebrospinal fluid, preferably with one or more agents capable of promoting penetration across the blood-brain barrier. In some embodiments, local administration of the agent is particularly useful.

Compositions for topical administration include, for example, sterile and non-sterile aqueous solutions, non-aqueous solutions in common solvents such as alcohols, or solutions in liquid or solid oil bases. Such solutions also can contain buffers, diluents, and other suitable additives. Compositions for topical administration can be formulated in the form of transdermal patches, ointments, lotions, creams, gels, drops, suppositories, sprays, liquids, and powders. Conventional pharmaceutical carriers, aqueous, powder or oily bases, thickeners, and the like can be added. Topical administration may be particularly useful for cutaneous diseases associated with granulomatous inflammation.

Compositions for oral administration include, for example, powders or granules, suspensions or solutions in water or non-aqueous media, capsules, sachets, or tablets. Such compositions also can incorporate thickeners, flavoring agents, diluents, emulsifiers, dispersing aids, or binders. Compositions for parenteral, intrathecal, or intraventricular administration can include, for example, sterile aqueous solutions, which also can contain buffers, diluents, and other suitable additives (e.g., penetration enhancers, carrier compounds, and other pharmaceutically acceptable carriers).

Methods described herein can include monitoring the patient, for example, to determine if granulomatous inflammation is improving with treatment. Any method can be used to monitor the patient. For example, granulomas can be examined to determine the number or types of cells such as macrophages, lymphocytes, fibroblasts, and giant cells that are present. The size, shape, and/or condition (e.g., fibrotic or necrotic) of granulomas also can be monitored to determine if the granuloma is resolving. In addition, in the case of infectious disease induced granulomatous inflammation (e.g., bacterial induced), the infection can be monitored to determine if the infection is resolving.

One or more agents having the ability to reduce B7-H1 or B7-H4 expression or activity can be combined with packaging material and sold as a kit for reducing granulomatous inflammation in a mammal (e.g., a human) or treating diseases associated with granulomatous inflammation. Components and methods for producing articles of manufactures are well known. For example, a kit can include antibodies that bind to a B7-H1 polypeptide (e.g., hB7-H1) and/or antibodies that bind to a B7-H4 polypeptide (e.g., hB7-H4). A kit also can include one or more antisense oligonucleotides or siRNA. The agents having the ability to reduce B7-H1 and/or B7-H4 expression can be in a container, such as a plastic, polyethylene, polypropylene, ethylene, or propylene vessel (e.g., a capped tube or a bottle). In addition, the articles of manufacture may further include reagents such as sterile water or pharmaceutical carriers for administering such agents to a mammal. Articles of manufacture also can include other agents useful for treating a patient (e.g., an antibiotic or other compound for treatment of diseases associated with bacterial induced granulomatous inflammation, an anti-fungal compound for treatment of diseases associated with fungal induced granulomatous inflammation, or a chemotherapy agent) in separate containers or admixed with agents having the ability to reduce B7-H1 and/or B7-H4 expression. Instructions describing how the various agents are effective for reducing granulomatous inflammation also may be included in such kits.

The invention will be further described in the following examples, which do not limit the scope of the invention described in the claims.

### EXAMPLE

Sixteen tumor recurrence specimens from BCG-refractory pTa/pT1 bladder tumors were assessed for PD-L1 staining. Briefly, paraffin-embedded tumor specimens were deparaffinized in xylene and rehydrated in a graded series of alcohols. Slides were unmasked in Target Retrieval Solution (DakoCytomation, Glostrup, Denmark) using a Decloaking Chamber (Biocare Medical, Walnut Creek, Calif.) and then blocked for endogenous peroxidase for 5 minutes with a peroxidase blocking solution. Slides were then rinsed in TRIS-buffered saline with 0.1% Tween 20 (TBST), incubated for 30 minutes with 1.5% normal horse serum in TBST, rinsed in TBST, and blocked for endogenous avidin and biotin. Slides were then incubated overnight at 4° C. with anti-PD-L1 (clone 5H1) at a concentration of 1:100. This step was followed by 30 minutes of incubation with biotinylated horse anti-mouse immunoglobulin G and avidin/biotin complex reagent. Slides were amplified using a Tyramide Signal Amplification Biotin System (Perkin-Elmer, Boston, Mass.) and incubated in 3-amino-9-ethylcarbazole chromogen. Isotype-matched antibodies were used to control for nonspecific staining.

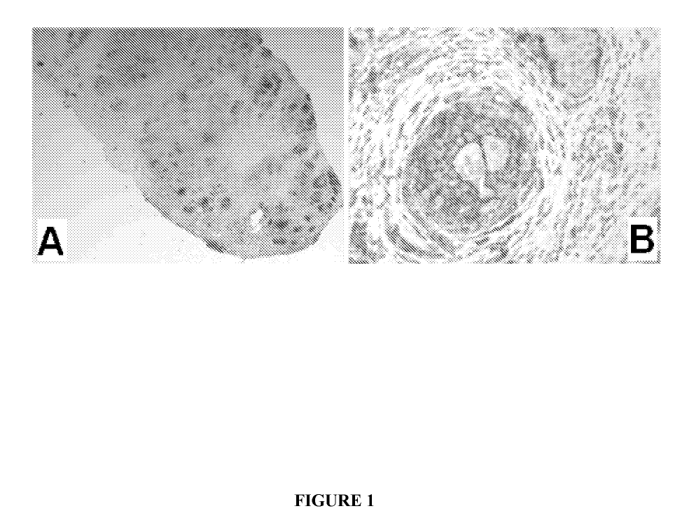

Twelve of the sixteen cases were found to have histologic BCG granulomas. Of these 12 cases, 11 had a very distinct pattern of widespread and intense PD-L1 staining that was primarily observed within BCG-induced granulomata (FIGS. 1A and 1B).

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method for reducing bacterial induced granulomatous inflammation in a mammal, said method comprising administering to said mammal an agent that reduces B7-H1 expression or activity.

2. The method of claim 1, wherein said agent is an antibody.

3. The method of claim 1, wherein said agent is an antisense oligonucleotide.

4. The method of claim 1, wherein said agent is a double-stranded small interfering RNA.

5. The method of claim 1, wherein said mammal is a human.

6. The method of claim 1, wherein the bacterial induced granulomatous inflammation is from a Mycobacterium infection.

7. The method of claim 6, wherein the bacterial induced granulomatous inflammation is from a Mycobacterium tuberculosis, Mycobacterium leprae, or Mycobacterium lepromatosis infection.

8. The method of claim 1, wherein said agent is administered locally to the granulomatous inflammation.

9. The method of claim 1, wherein the bacterial induced granulomatous inflammation is from a Mycobacterium bovis strain bacille Calmette-Guérin (BCG) infection.

10. The method of claim 1, further comprising monitoring said patient to determine if granulomatous inflammation is improving with treatment.

11. A method for reducing bacterial induced granulomatous inflammation in a mammal, said method comprising administering to said mammal an agent that reduces B7-H4 expression or activity.

12. The method of claim 11, wherein said agent is an antibody.

13. The method of claim 11, wherein said agent is an antisense oligonucleotide.

14. The method of claim 11, wherein said agent is a double-stranded small interfering RNA.

15. The method of claim 11, wherein said mammal is a human.

16. The method of claim 11, wherein the bacterial induced granulomatous inflammation is from a Mycobacterium infection.

17. The method of claim 16, wherein the bacterial induced granulomatous inflammation is from a Mycobacterium tuberculosis, Mycobacterium leprae, or Mycobacterium lepromatosis infection.

18. The method of claim 11, wherein said agent is administered locally to the granulomatous inflammation.

19. The method of claim 11, wherein the bacterial induced granulomatous inflammation is from a Mycobacterium bovis strain BCG infection.

20. The method of claim 11, further comprising monitoring said patient to determine if granulomatous inflammation is improving with treatment.
